(19) United States
(12) Patent Application Publication
(10) Pub. No.: US 2009/0208417 A1
(43) Pub. Date: Aug. 20, 2009

(54) Detection and Localized Imaging of Cancer Using X-Ray Fluorescent Nanoparticle/Preferential Locator Conjugates

(75) Inventors: Marlin O. Thurston, Columbus, OH (US); Vishwanath V. Subramaniam, Westerville, OH (US)

Correspondence Address: MUELLER AND SMITH, LPA;MUELLER-SMITH BUILDING, 7700 RIVERS EDGE DRIVE, COLUMBUS, OH, 43235, US

(73) Assignees: Actis, Ltd, Columbus, OH (US); The Ohio State University, Columbus, OH (US)

(21) Appl. No.: 12/365,939

(22) Filed: Feb. 5, 2009

Related U.S. Application Data
(60) Provisional application No. 61/029,402, filed on Feb. 18, 2008.

(51) Int. Cl. A61K 49/08

(52) U.S. Cl. 424/9.4

(57) ABSTRACT

A method for detecting gold or other nanoparticles conjugated to preferential locators commences by contacting tissue suspected of being neoplastic with gold nanoparticle/preferential locator conjugates for a time adequate for the conjugates to bind with the tissue. A beam of gamma photons (such as from, ^{99m}Tc) is directed at the conjugate bound tissue to remove electrons from the K-shell or L-shell, for example, of the gold nanoparticles. The removed electrons can be detected for locating neoplastic tissue or X-ray fluorescence corresponding to an electron transitioning from one shell to another shell can be detected, or by detecting resulting X-ray fluorescence corresponding to an electron transitioning from one shell to another shell, such as X-ray fluorescence arising from K-alpha emission corresponding to an electron transitioning from the L shell to the K shell. Additional X-ray fluorescing detecting molecules include Ag, I, Fe, Tc, Zn, Mn, Cr (trivalent).

### CROSS-REFERENCE TO RELATED APPLICATIONS

This application claims benefit of provisional application Ser. No. 61/029,402, filed Feb. 16, 2008.

### STATEMENT REGARDING FEDERALLY SPONSORED RESEARCH

Not applicable.

### BACKGROUND

The present disclosure generally relates to cancer and more particularly to locating and differentiating cancer using gold nanoparticles and other X-ray fluorescence detecting molecules.

Tumor-associated antigen (TAG-72) is a human mucin-like (MUC1) glycoprotein complex with molecular weight of 10⁶ Da. It is over-expressed in several epithelial-derived cancers, including most ductal carcinomas of the breast, common epithelial ovarian carcinomas, non-small cell lung carcinomas, gastric, pancreatic, and colorectal carcinomas. Murine monoclonal antibody (B72.3) was generated using membrane-enriched extracts of human metastatic mammary carcinoma lesions, while the second generation monoclonal antibody (CC49) was generated against purified TAG-72 from colon cancer. These antibodies have been extensively evaluated in animal models and human for detection of various cancers, one of which has been approved by FDA for the detection of both colorectal and ovarian cancers with gamma camera scanning in conjunction with computerized tomography (¹¹¹Indium labeled B72.3 antibody, CYT-103, Cytogen).

TAG-72 antibody shows selective reactivity for human carcinomas, demonstrating that 94% of colon carcinomas express the TAG-72, while normal colon epithelium does not show any reactivity to the antibody. Murine monoclonal B72.3 also reacted with cells in areas of “atypia” within adenomas. It also showed reactivity with other human carcinomas including 84% of invasive ductal breast cancer, 100% of ovarian cancers tested, and 96% of lung of adenocarcinomas, while it showed only weak or no reactivity in the corresponding normal tissues except secretory endometrium.

B72.3 antibody has been evaluated in tissue culture and xenograft models. Interestingly, this antibody is not reactive to vast majority of human carcinoma cell lines in cultures due to limitations in this special configuration. However, it is highly expressed in colon cancer cell lines (e.g., LS 174T) and breast cancer cells lines (e.g., MCF-7). When these cells were grown in spheroid culture, suspension cultures or on agar, TAG-72 expression increased by 2-10 fold. Additionally, when the LS 174T cell line was injected into athymic mice to generate xenograft models, the level of TAG-72 antigen increased over 100-fold, which is similar to expression levels seen in the metastatic tumor masses from patients. I¹²⁵-labeled B72.3 was tested in xenograft mice models with LS-174 cancer cells for tumor localization. After intravenous injection of 1.5 μCi of ¹²⁵I-labeled B72.3, 10% of injected dose per gram of body weight (% ID/g) was determined after two days. Interestingly, the total amount of ¹²⁵I-B72.3 activity in the tumor stayed constant during 30 days, while the activity in the rest of the body including blood, kidney, liver, spleen, and lung decreased significantly. For example, The % ID/g of ¹²⁵I-B72.3 in tumors stayed at 6.49% to 10.75% in 7 days period, while it decreased from 9.94% to 1.38% in blood, 1.82% to 0.34% in kidney, 2.23% to 0.37% in spleen, 5.52% to 0.75% in lung, and 1.89% to 0.37%. The distribution ratio of tumor compared to other normal organs (liver, kidney, lung) reached 18:1 at day 7, while tumor to blood ratio reached 5:1 at day 7. In xenograft models with A375 cells without TAG-72 expression, B72.3 did not show any tumor localization. In xenograft models implanting LS 174T with high levels of TAG-72, other control antibodies such as ¹²⁵I-MOPC-21 IgG did not show tumor localization either.

¹³¹I labeled B72.3 IgG has been used clinically for diagnostic imaging of colorectal, ovarian, and breast cancer. The data demonstrate the specific localization of B72.3 antibody in cancer tissues in patients. After intravenous (IV) administered ¹³¹I-labeled B72.3 IgG prior to surgery, radio-localization indices (RI) were calculated by cpm (counts per minute) of ¹³¹I-labeled antibody per gram of tumor versus cpm per gram of normal tissues. Seventy percent (99 of 142) of tumor lesions showed RI greater than 3 (antibody localization in tumors is 3 times greater than normal tissue). In addition, high-performance liquid chromatography (HPLC) and SDS-polyacrylamide gel electrophoresis demonstrated that the radioactivity in patient's sera was associated with intact ¹³¹I-B72.3 antibody as visualized in autoradiography or IgG peak in HPLC analysis after IV administration of dose range 0.5-20 mg. Interestingly, when ¹³¹I-labeled B72.3 IgG was administered intraperitoneally in colon cancer patients, the localization in colon tumor verse normal tissue was 70:1. However, IV administration of this labeled antibody is more efficient in targeting lymph node metastases.

¹²⁵I-labeled B72.3 also has been used for radio-immunoguided surgery (RIGS®, U.S. Pat. No. 4,782,840) with an intraoperative hand-held probe to localize the residual tumor tissue for resection. The RIGS system also has been successfully used with the B72.3 antibody for clinical colorectal cancer patients. ¹²⁵I labeled-antibody has localized 75%-80% of primary colorectal tumor lesion, and 63%-73% of metastatic lesions in lymph nodes and liver.

The second-generation antibody CC49 was generated against TAG-72 purified from colon cancer. CC49 showed higher binding affinity than B72.3 to TAG-72 in carcinomas including breast, colorectal, ovarian, and lung carcinomas, while CC49 exhibited minimum reactivity with normal tissues. When ¹²⁵I-CC49 was administered in xenograft models with colon cancer cells LS 174T, the plasma clearance was much faster than B72.3, which results in much higher tumor to normal tissue distribution ratio. For example, the tumor to blood ratio was 18.1, tumor to liver ratio 3.81, tumor to spleen ratio 16.64, tumor to kidney ratio 36.48, and tumor to lung ratio 25.82. In RIGS studies of 300 patients with colorectal cancers, CC49 was able to successfully detect tumors in 86% of patients with primary tumors and 95% of patients with recurrent tumors. In addition, clinical studies of a modified humanized antibody CC49ΔC_{H}2 with a deletion in glycosylation sites of the antibody showed similar results with CC49 in detection of colorectal cancer.

Both B72.3 and CC49 have demonstrated promising results in tumor detection utilizing the RIGS procedure to significantly improve patient survival rate. However, in many cases, patients have shown metastatic cancers or multiple lesions, which are not resectable. In such cases, even though the antibodies used with RIGS are able to detect the tumors, surgery cannot be employed to remove the tumors. The long half-life of ¹²⁵I, waste disposal of ¹²⁵I, and other problems associated with ¹²⁵I also make this procedure difficult for the market to accept. Other labels, such as, for example, ¹⁸F with a 110-minute half life, will not work in this procedure, because of the need to wait 21 days after antibody injection in order for non-bound antibody to clear the body.

Thus, there exists a need for new (non-radioactive) molecules for use with the foregoing MAbs, as well as for other neoplastic tissue preferential locators.

### BRIEF SUMMARY

A method for detecting gold nanoparticles conjugated to preferential locators commences by contacting tissue suspected of being neoplastic with gold nanoparticle/preferential locator conjugates for a time adequate for the conjugates to bind with the tissue. A beam of gamma photons (such as from, ^{99m}Tc) is directed at the conjugate bound tissue to remove electrons from the K-shell of the gold nanoparticles. The removed electrons can be detected for locating neoplastic tissue or X-ray fluorescence corresponding to an electron transitioning from one shell to another shell can be detected, or by detecting resulting X-ray fluorescence corresponding to an electron transitioning from one shell to another shell, such as X-ray fluorescence arising from K-alpha emission corresponding to an electron transitioning from the L shell to the K shell.

In a similar manner, additional X-ray fluorescence detecting molecules can similarly be conjugated with preferential locators and used in a manner similar to the disclosed Au conjugates. Additional such X-ray fluorescence detecting molecules include, for example, I, Ag, In, Tc, Mo, Bi, Eu, Tb, and the like.

### BRIEF DESCRIPTION OF THE DRAWINGS

For a fuller understanding of the nature and advantages of the present device, process, and apparatus, reference should be had to the following detailed description taken in connection with the accompanying drawings, in which:

FIG. 1 is a simplified reaction schematic showing a two-step EDC/suflo-NHs mediated amine and carboxyl coupling reaction for labeling CC-49 antibody with gold nanoparticle;

FIG. 2 is a simplified schematic showing disclosed method where the antibody conjugated gold nanoparticles are injected into a patient, suspected neoplastic tissue removed, and the PIXI (Photon Induced X-ray Imaging) Margin Analyzer used to confirm tumor margins real-time and intraoperatively;

FIG. 3 is a plot of X-ray fluorescence of bulk 22-carat gold induced by gamma-ray excitation from a ⁵⁷Co source (5 mCi activity);

FIG. 4 is a plot of probability versus distance showing depth of penetration of X-rays and gamma-rays of various energies;

FIG. 5 is a plot of X-ray fluorescence from 14K gold excited by 140 keV photons from a 1.8 mCl ^{99m}Tc source; and

FIG. 6 is a simplified schematic showing Photon Induced X-ray Intraoperative Detection (PIXID) of cancer.

The drawings will be further elaborated below.

### DETAILED DESCRIPTION

The fundamental idea is to perform cancer diagnosis and detection with neoplastic tissue preferential locators, such as, for example, cancer-specific antibodies, that have been labeled with a detectable non-radioactive atom that has a relatively high atomic number. In diagnostic practice, for example, the antibodies, preferably monoclonal antibodies (MAbs) are injected into the blood stream of the patient and, after a period of time, preferentially accumulate in malignant sites. Exciting K-shell x-ray emission from the labels and detecting such excitation, then, identify the sites. The excitation is produced by a directed beam of gamma photons that have a sufficiently high energy to remove electrons from the K-shell of the label. The location of the malignant site (neoplastic tissue or cancer, for example) that emits the K-shell x-rays, then, can be determined with a suitable probe or x-ray imaging device.

This technique has the advantage compared to, for example, mammography, computed tomography (CT) scans, ultrasonic scans, and positron emission tomography (PET) scans, of being cancer specific. Also, this technique has the advantage, compared to various gamma camera techniques, of not requiring the injection of radioactive agents into the patient's blood stream.

Implementation of this technique, for example, involves injection of the antibody-gold nanoparticle complex. The K-shell excitation of gold is 80.725 keV and K-alpha x-rays have energies of 66.4, 67.0, and 68.8 keV. Gold nanoparticles have regulatory approval for human use and are commercially available in the range of sizes appropriate for conjugation to antibodies. A useful excitation is the 140 keV gamma radiation of technetium-99m (^{99m}Tc). Since ^{99m}Tc is used in stress tests and numerous other medical applications, it is available in most hospitals.

Binding energies for the other fluorescence detecting molecules are as follows: I (33.169 keV); In (27.94 keV); Tc (21.044 keV); Mo (20.00 keV); Bi (90.526 keV); Eu (48.519 keV); and Tb (51.996 keV). Excitation sources will be adjusted accordingly. Several of these additional fluorescence-detecting molecules already are in commercial use, including for human use.

Eu, for example, can be configured as a chelate designed for peptide and protein labeling in aqueous solution phase. See, for example, Org Biomol Chem. 2006 Nov. 21; 4(22):4165-77; Bioconjug Chem. 2001 November-December; 12(6):845-9; Anal Chem. 1998 Feb. 1; 70(3):596-601; Zh Mikrobiol Epidemiol Immunobiol. 1994 July-August; (4):59-63; J Immunol Methods. 1993 May 5; 161(1):1-6; Eur J Clin Chem Clin Biochem. 1992 September; 30(9):529-30; and Clin Biochem. 1988 June; 21(3):173-8.

The detector could be configured as a hand held probe coupled with a source of gamma radiation. A beam-forming technetium source can be as simple as a tube of, for example, silver-tin (Ag—Sn) alloy with a wall thickness of, say, about 2 or 3 mm. The source is placed on the inside of a plug at the back end, and radiation is restricted to a narrow beam by a plug at the front end with an aperture of, say, about 5 mm diameter. The detector probe may be collimated to provide a rough indication of target depth or it may be uncollimated for greater sensitivity.

Laparoscopic and endoscopic applications may be limited due to shielding issues, but should not be ruled out based on unique instrument designs. The 1/e distance for gold K-alpha radiation in tungsten is about 0.3 mm and comparable for silver-tin alloys.

### Preferential Locators

Such “locator” includes a substance, which preferentially concentrates at the tumor sites by binding with a marker (the cancer cell or a product of the cancer cell, for example) produced by or associated with neoplastic tissue or neoplasms. Appropriate locators today primarily include antibodies (whole and monoclonal), antibody fragments, chimeric versions of whole antibodies and antibody fragments, and humanized versions thereof (see Background section, above), somatostatin congeners (see U.S. Pat. No. 5,590,6560), and similar molecules.

Thus, antibody CC49, its humanized and domain deleted forms, and related TAG antibodies have been described in the literature, such as, by Xiao, et al., “Pharmocokinetics and clinical evaluation of ¹²⁵I-radiolabeled humanized CC49 monoclonal antibody (HuCC49ΔC_{H}2) in recurrent and metastatic colorectal cancer patients”, Cancer Biother Radiopharm, vol. 20, number 1, 2005; Fang, et al., “Population pharmacokinetics and tumor targeting of HuCC49ΔCH2, a novel monoclonal antibody for tumor detection”, Fang, et al., J Clin Pharmacol 2007; 47:227-237; U.S. Pat. Nos. 6,418,338 and 6,760,612 (which also show peptide, lectin, and other detector molecules. See also, Slavin-Chiorini, et al., “A CDR-Grafted (Humanized) Domain-Deleted Antitumor Antibody”, Cancer Biotherapy and Radiopharmaceuticals, Volume 12, Number 5, 1997, Mary Ann Liebert, Inc. (“The MAb chosen for engineering was CC49, which is directed against a pancarcinoma antigen designated TAG-72 that is expressed on the majority of colorectal, gastric, breast, ovarian, prostate, pancreatic and lung carcinomas.”).

Yet another humanized antibody of CC49 MAb is known as V59. Gonzales, et al., “Minimizing immunogenicity of the SDR-grafted humanized antibodyCC49 by genetic manipulation of the framework residues”, Molecular Immunology 40 (2003) 337-349. V59 is reported to be a fully humanized version of CC49 MAb, making it a likely choice for use in accordance with the disclosure set forth herein.

In the early 1990s investigators utilized the RIGS system to locate, differentiate and stage other types of cancer, for instance, endocrine tumors involved, inter alia, with breast, gastrinomas, lung, and nervous system. Generally, the approach was to administer a radiolabeled somatostatin congener to assess the patient with the RIGS probe. However, before subjecting the patient to such administration, an initial determination preferably was made as to whether the radiolabeled somatostatin congener would bind to the tumor site, i.e., whether somatostatin receptors are associated with the neoplastic tissue. This was conveniently done with a wide variety of endocrine tumors, which release peptides or hormones, referred to as “biochemical markers.” In order to make this determination, initially a biochemical marker-inhibiting dose of unlabeled somatostatin congener was administered to the patient. The biochemical marker associated with the neoplastic tissue then was monitored to determine whether the administered somatostatin congener reduces the presence of the marker in the patient. If the monitored presence of the marker was reduced, then the surgeon could be confident that the neoplastic tissue or tumor contains receptors to which the somatostatin would bind. Thus, the administration of radiolabeled somatostatin congener was appropriate for such patient. If the biochemical marker associated with the neoplastic tissue was not appropriately reduced following the administration of the unlabeled somatostatin congener, then the neoplastic tissue may not be determinable by the use of radiolabeled somatostatin congener and alternative modalities of treatment would be considered, such as the use of radiolabeled antibodies. See: O'Dorisio, et al., U.S. Pat. No. 5,590,656; entitled “Application of Peptide/Cell Receptor Kinetics Utilizing Radiolabeled Somatostatin Congeners in the In Situ, In Vivo Detection and Differentiation of Neoplastic Tissue”; issued Jan. 7, 1997 and incorporated herein by reference.

In broader contexts, a locator that specifically binds a marker produced by or associated with neoplastic tissue is used in accordance with the present teachings, with antibodies and somatostatin congener being representative such locators. Broader, however, a “locator” includes a substance that preferentially concentrates at the tumor sites by binding with a marker (the cancer cell or a product of the cancer cell, for example) produced by or associated with neoplastic tissue or neoplasms. Appropriate locators today primarily include antibodies (whole and monoclonal), antibody fragments, chimeric versions of whole antibodies and antibody fragments, and humanized versions thereof. It will be appreciated, however, that single chain antibodies (SCAs, such as disclosed in U.S. Pat. No. 4,946,778, incorporated herein by reference) and like substances have been developed and may similarly prove efficacious. For example, genetic engineering has been used to generate a variety of modified antibody molecules with distinctive properties. These include various antibody fragments and various antibody formats. An antibody fragment is intended to mean any portion of a complete antibody molecule. This includes both terminal deletions and protease digestion-derived molecules, as well as immunoglobulin molecules with internal deletions, such as deletions in the IgG constant region that alter Fc mediated antibody effector functions. Thus, an IgG heavy chain with a deletion of the Fc CH2 domain is an example of an antibody fragment. It is also useful to engineer antibody molecules to provide various antibody formats. In addition to single chain antibodies, useful antibody formats include divalent antibodies, tetrabodies, triabodies, diabodies, minibodies, camelid derived antibodies, shark derived antibodies, and other antibody formats. Aptamers form yet a further class of preferential locators. All of these antibody-derived molecules are example of preferential locators.

In addition to antibodies, biochemistry and genetic engineering have been used to produce protein molecules that mimic the function of antibodies. Avimers are an example of such molecules. See, generally, Jeong, et al., “Avimers hold their own”, Nature Biotechnology Vol. 23 No. 12 (December 2005). Avimers are useful because they have low immunogenicity in vivo and can be engineered to preferentially locate to a wide range of target molecules such as cell specific cell surface molecules. Although such substances may not be subsumed within the traditional definition of “antibody”, avimer molecules that selectively concentrate at the sites of neoplastic tissue are intended to be included within the definition of preferential locator. Thus, the terms “locator” was chosen, to include present-day antibodies and equivalents thereof, such as avimers, as well as other engineered proteins and substances, either already demonstrated or yet to be discovered, which mimic the specific binding properties of antibodies in the inventive method disclosed therein.

Labeling of Humanized MAb with Gold NPs

Gold nanoparticles (NPs) coupled to active esters, amines, and maleimides are commercially available. These reagents have been coupled to antibodies (other than CC-49) and other proteins, using essentially the same methods as used, for example, for the cyanine fluorochromes, though there are some batch-to-batch variations in coupling efficiency. Conjugates are characterized by gel electrophoresis, using, for example, a silver staining protocol specific for gold to identify the conjugates (Li Silver, Nanoprobes, Inc.).

Cy7-CC-49-1 has been conjugated with gold NPs. Water-soluble gold NPs have little selectivity to target tumor tissues by themselves, and need to be attached to bio-affinity ligands, such as antibodies, which can specifically bind to antigen on the surface of tumor cells. The most popular conjugation approach is direct amide formation between gold NPs and antibody through carboxylate-amine condensation using carbodiimide (EDC) and sulfo-NHS[12-14]. Most biomolecules such as IgGs, contain primary amines and negatively charged gold NPs contain carboxyl groups on their surface. The antibody and gold NPs, therefore, can be directly coupled through carboxylate-amine condensation. The advantage of this approach is that EDC and sulfo-NHS are zero-length cross-linking agents and the conjugation will not increase the size of conjugates.

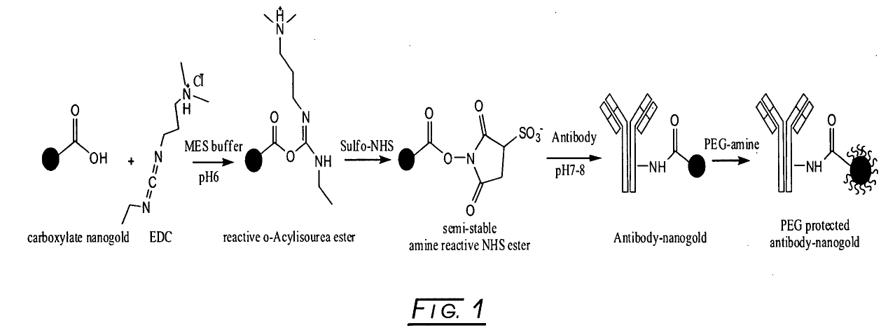

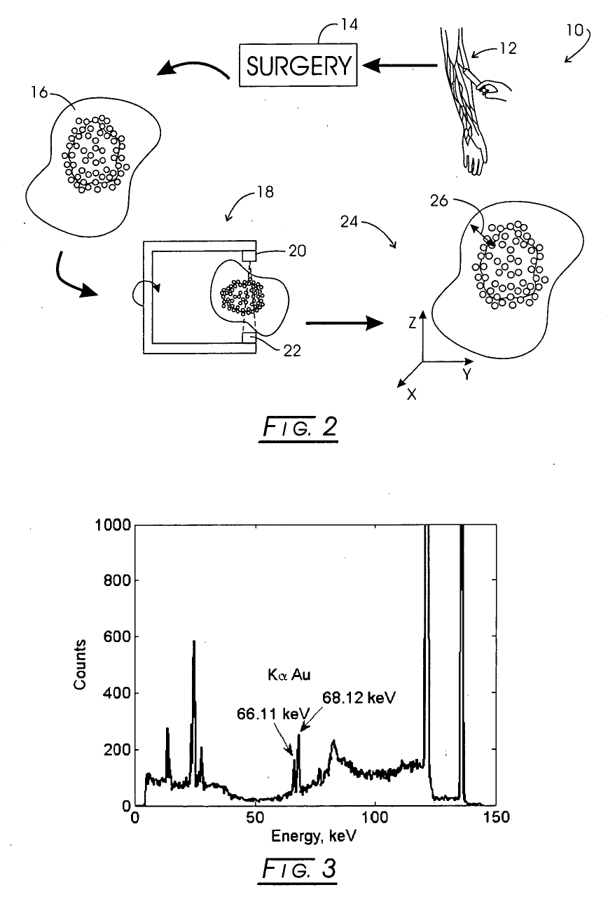

A two-step EDC/sulfo-NHS mediated amine and carboxyl coupling reaction protocol has been well established in studies of the current technique. As shown in FIG. 1, in the first step, the carboxyl groups (18 carboxyl groups/gold NP) are activated by EDC to form a reactive o-acylisourea ester at pH 6 in 0.1 M MES buffer solution. However, the o-acylisourea ester is unstable in aqueous solution and, therefore, sulfo-NHS is required to form a semi-stable sulfo-NHS ester (sulfo-NHS esters in aqueous solutions have a half-life of 4-5 hours at pH 7, 1 hour at pH 8 and only 10 mins at pH 8.6), resulting in a higher coupling efficiency. Meanwhile, activation with sulfo-NHS increases the solubility of the gold NPs and prevents their aggregation. An optimum concentration of EDC and sulfo-NHS, respectively, seems to be 2 mM and 5 mM. At room temperature, the gold NPs are activated in 15 minutes.

In the second step, the reaction of sulfo-NHS activated gold NPs with primary amines is most efficient at pH 7-8 in phosphate-buffered saline (PBS). A PBS equilibrated spin desalting column (Pierce) is used to exchange the MES buffer into PBS buffer and remove excessive EDC and sulfo-NHS within 3 minutes. Otherwise, the excessive EDC and sulfo-NHS will activate the carboxyl groups of the antibody. The eluted sulfo-NHS activated gold NP solution is at pH 7 in PBS and then is added to the Cy7-CC-49 solution. To accelerate the reaction, 0.01 M Na₂CO₃ may be used to adjust the pH to 8. After 2 hours of reaction, excessive PEG-amines (MW 5000) are added to react with the unreacted sulfo-NHS esters on the surface of the gold NPs. Otherwise, the unreacted sulfo-NHS esters will be hydrolyzed, thereby generating the original carboxyl groups. The free carboxyl acid on the surface of the gold NPs will cause non-specific binding between gold NP-antibody conjugates and plasma proteins, resulting in clearance of the conjugates via the reticuloendothelium system.

Finally, the conjugate is purified with a Superdex-200 gel column. The conjugates (135-165 kD) will elute first (the first brown band), while the unbound gold NPs (15 kD) and unreacted PEG-amines (5 kD) will elute later. The conjugate solution is collected and concentrated in a 30 kD cut-off centrifuge filter (Millipore, Billerica, Mass.). The concentration of gold NP-CC-49-Cy7 conjugate and Cy7/gold NP/CC-49 ratio can be estimated by UV-Vis assay.

Once the antibodies (CC-49) have been conjugated with a label, they will be injected into the patient for imaging and detection. The problem, thus, is reduced to imaging or detecting the label (gold NP). Such detection can be practiced for intraoperative detection of cancerous tissue (in vivo detection) or for determining/confirming tumor margins of excised tissue (ex-vivo detection).

### Photon Induced X-Ray Imaging (PIXI) of Excised Tissue for Margin Analysis

A question frequently asked by patients, their families, and even the surgical oncologists themselves, is whether or not surgical resection was successful in removing all cancerous tissue. The state-of-the-art method of determining surgical margins involves visual inspection by the surgeon along with optical microscope analysis of select miniscule tissue specimens by the pathologist. Unfortunately, pathological exploration of sections of excised tissue is an inaccurate and unreliable means of assessing margins since it is partial, incomplete, and dependent to a large degree on the individual pathologist. Hence, a negative result from pathological examination is at best inconclusive. More recently micro PET/CT imaging has been used, but current equipment is too large and bulky for intraoperative use.

The disclosed technique can be used to determine/confirm tumor margins using a process similar to that used intraoperatively. FIG. 3 shows a photon induced X-ray emission spectrum of bulk 22K gold. Excitation of the fluorescence was accomplished using gamma-rays from a low-level source (⁵⁷Co, 5 mCi). This spectrum was obtained using a tin collimator and liquid nitrogen cooled Germanium (Ge) detector, with scattering at 90°. An important difference between existing, commercially available X-ray Fluorescence (XRF) and Energy Dispersive X-ray Spectroscopic (EDS) analysis instruments, and PIXI is that in the latter, a low-level, safe radioisotope is used as the excitation source instead of a bulky X-ray tube and associated power supply. PIXI also may employ computed tomographic (CT) techniques to generate a 3-D image of the gold NPs attached to the cancer within the excised tissue. Displayed graphically on a computer, this will provide the surgeon with real-time, intraoperative information regarding the extent of the cancer within the surgically excised tissue and help determine whether more tissue needs to be resected.

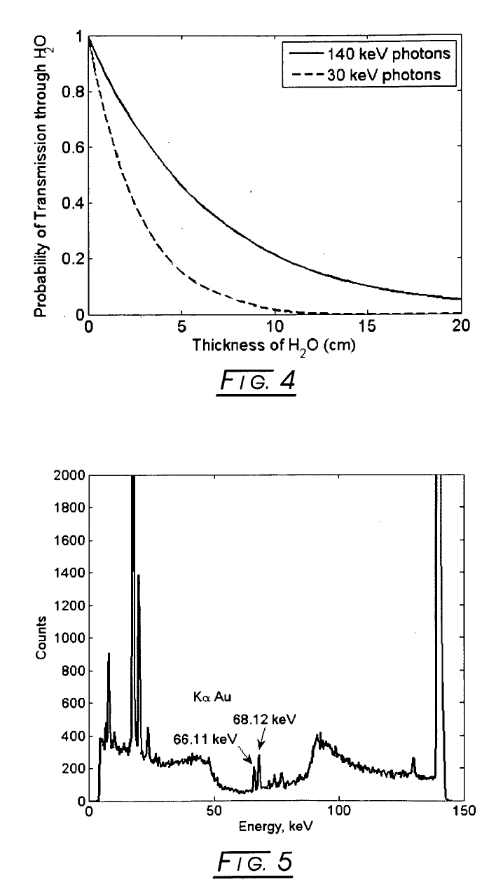

An important advantage of using X-rays and the PIXI technique over any optical technique such as near-IR fluorescence is the deep penetration depth, enabling imaging of embedded tumors. Shown in FIG. 4 is the depth of penetration of X-rays and gamma-rays of various energies through water, and hence to a good approximation, through biological tissue. As can be seen, photons having energy of 140 keV (corresponding to gamma-ray emission from ^{99m}Tc) are able to penetrate many centimeters into tissue, as are X-ray photons corresponding to the K-alpha emission in gold. In detecting the presence of conjugated gold NPs, it is anticipated that a good level of signal may be obtained because of the accumulation of these particles in the vicinity of cancer cells. Moreover, detection of conjugated gold NPs may be more easily enabled by using a stronger radioisotope that is housed in a well shielded table-top instrument.

### Photon Induced X-Ray Intraoperative Detection (PIXID) of Cancer

Excitation of X-ray fluorescence from conjugated gold NPs using a suitable gamma-ray source such as ^{99m}Tc or ⁵⁷Co, can be used to detect the gold NPs in vivo. FIG. 5 shows an X-ray fluorescence spectrum from 14K gold excited by the 140 keV gamma-rays of ^{99m}Tc. The operating principle is the same as that of the PIXI Margin Analyzer described above, except that detection can be accomplished with far less signal. The presence of the conjugated gold NPs, an indication of cancer, can be detected by comparing the X-ray fluorescence signal at a given location with the corresponding signal at another location where there are no cancer cells. Such a comparison allows signal levels as low as a few counts per second above background to be used to detect the presence of cancer. There is precedence for in vivo detection of elemental gold administered in the form of gold salts and without conjugation to a preferential locator, in human patients undergoing chrysotherapy for treatment of rheumatoid arthritis [J. Shakeshaft and S. C. Lillicrap, The British Journal of Radiology 66, pp. 714-717, 1993]. However, without antibodies and without gold NPs, acceptable signal was only obtained using a strong (300 mCi) ¹⁵³Gd external source.

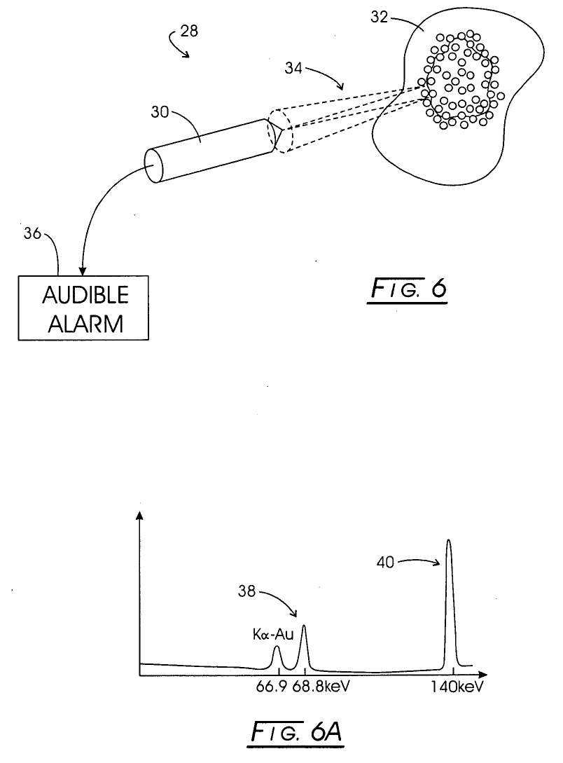

Intraoperative detection may be accomplished in more than one way. FIG. 6 shows a schematic of a backscatter geometry. In this configuration, a handheld probe housing the ^{99m}Tc source and detector may be used by the surgeon to detect the conjugated gold NPs that have previously been allowed to accumulate in the vicinity of the tumor. While ergonomically appealing, this configuration is likely to suffer from the adverse effects of Compton scattering. Compton scattering is the elastic scattering of a photon off an electron. The wavelength of the scattered photon when colliding with a free electron, is shifted according to the following equation:

$${λ}^{′} - λ = \frac{h}{{m}_{0} c} ( 1 - cos φ )$$

where φ is the angle between the direction of the scattered photon and the incident photon. In the backscatter geometry, φ≈180°, so that the wavelength shift is maximum. Additional Compton scattering off loosely bound electrons and electrons existing within the detector compound the problem. The resulting increase in background signal can couch the desired fluorescence signal from the gold.

An alternative approach is to house just the radioisotope in the handheld probe so as to produce a collimated beam of 140 keV gamma-rays. An array of detectors then is embedded within the operating table on which the patient is laid. X-ray fluorescence from the gold NPs will then be detected in transmission so that Compton scattering is minimized (since in transmission, φ=0).

While the devices and process have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

We claim:

1. A method for detecting X-ray fluorescence emitting molecules conjugated to preferential locators, which comprises the steps of:
(a) introducing a X-ray fluorescence emitting molecule/preferential locator conjugate into a patient suspected of having neoplastic tissue;
(b) permitting time to elapse to permit said conjugate to bind with said suspect tissue and for unbound conjugate to be cleared;
(c) directing a beam of gamma ray photons at the suspect neoplastic tissue; and
(d) directing an X-ray fluorescence detector at said suspect neoplastic tissue to detect shell-transitioning electrons indicative of said conjugate being bound to said suspected neoplastic tissue or to detect X-ray fluorescence from shell transitioning electrons in nearby materials arising from excitation of photons emitted by said conjugate being bound to said suspected neoplastic tissue.

2. The method of claim 1, wherein said preferential locator is a monoclonal antibody.

3. The method of claim 2, wherein said monoclonal antibody binds tumor-associated antigen (TAG-72).

4. The method of claim 2, wherein said monoclonal antibody is one or more of B72.3 and CC49 monoclonal antibody, derivatives thereof, or humanized derivatives thereof.

5. The method of claim 4, wherein said monoclonal antibody is one or more of V59, CC49ΔCH₂, or HuCC49ΔCH₂.

6. The method of claim 1, wherein conjugate binds antigen associated with said neoplastic tissue in lymph tissue.

7. The method of claim 1, wherein said preferential locator is an aptamer.

8. The method of claim 1, wherein said preferential locator is an avimer.

9. The method of claim 1, wherein said preferential locator is a somatostatin congener.

10. The method of claim 1, wherein between steps (b) and (c), said suspect tissue is removed and then steps (b) and (c) are practiced in order to confirm the absence of neoplastic tissue at margins of said removed tissue.

11. The method of claim 10, wherein said margins are determined by photon induced X-ray imaging.

12. The method of claim 1, wherein said X-ray fluorescence detecting molecule is one or more of Au, Ag, Fe, I, Tc, Zn, Mn, Cr (trivalent).

13. A method for detecting gold nanoparticles conjugated to preferential locators, which comprises the steps of:
(a) introducing a gold nanoparticle/preferential locator conjugate into a patient suspected of having neoplastic tissue;
(b) permitting time to elapse to permit said conjugate to bind with said suspect tissue and for unbound conjugate to be cleared;
(c) directing a beam of gamma photons at the suspect neoplastic tissue; and
(d) directing an X-ray fluorescence detector at said suspect neoplastic tissue to detect shell-transitioning electrons indicative of said conjugate being bound to said suspected neoplastic tissue.

14. The method of claim 13, wherein said preferential locator is a monoclonal antibody.

15. The method of claim 14, wherein said monoclonal antibody binds tumor-associated antigen (TAG-72).

16. The method of claim 14, wherein said monoclonal antibody is one or more of B72.3 and CC49 monoclonal antibody, derivatives thereof, or humanized derivatives thereof.

17. The method of claim 16, wherein said monoclonal antibody is one or more of V59, CC49ΔCH₂, or HuCC49ΔCH₂.

18. The method of claim 13, wherein conjugate binds antigen associated with said neoplastic tissue in lymph tissue.

19. The method of claim 13, wherein said preferential locator is an aptamer.

20. The method of claim 13, wherein said preferential locator is an avimer.

21. The method of claim 13, wherein said preferential locator is a somatostatin congener.

22. The method of claim 13, wherein between steps (b) and (c), said suspect tissue is removed and then steps (b) and (c) are practiced in order to confirm the absence of neoplastic tissue at margins of said removed tissue.

23. The method of claim 22, wherein said margins are determined by photon induced X-ray imaging.
